(19) United States
(12) Patent Application Publication
(10) Pub. No.: US 2012/0083811 A1
(43) Pub. Date: Apr. 5, 2012

(54) Microkeratome system for the generation of a flap on the human eye

(76) Inventors: Christof Donitzky, Eckental/Eschenau (DE); Tobias Jeglorz, Nurnberg (DE)

(21) Appl. No.: 12/894,289

(22) Filed: Sep. 30, 2010

(51) Int. Cl. A61F 9/007

(52) U.S. Cl. 606/166

(57) ABSTRACT

A microkeratome system for the generation of a flap on the human eye includes a plurality of functionally different, separately interchangeable components with, in each instance, at least one characteristic feature. In addition, the system includes a control unit for controlling the operation of the microkeratome system, the control unit having access to stored information that is representative of at least one predetermined combination of the characteristic features of at least a fraction of the components, and also an input device for entering information that is representative of a desired combination of the characteristic features of at least a fraction of the components. The control unit is adapted to compare the desired combination of features with the at least one predetermined combination of features and to bring about a predetermined response, depending on the result of the comparison. In this way, the user can have his/her attention drawn to risky combinations, without having to rely solely on the wealth of experience of the user in connection with the selection of the components.

The invention relates to a microkeratome system for the generation of a flap on the human eye.

### BACKGROUND OF THE INVENTION

For the treatment of visual defects of the human eye, LASIK (laser in-situ keratomileusis) is a widespread technique. In this case a small disc, generally designated in specialist circles as a flap, is cut out on the surface of the cornea, said disc not being completely separated from the residual corneal tissue but being connected to the remaining corneal tissue via a so-called hinge. In this way, the flap can be easily folded aside in order to expose the underlying corneal tissue for an ablating laser treatment. After the laser treatment has taken place, the flap is folded back again; the preceding resection of corneal material then results in a correspondingly altered shape of the corneal surface, which at best fully compensates the visual defect to be eliminated.

In the case of classical LASIK, the flap is prepared by means of a mechanical microkeratome. In this case it is a question of a microsurgical plane with a razor-sharp cutting blade which is customarily driven in oscillating manner. An exemplary microkeratome system is marketed by WaveLight GmbH under the trade name RONDO; a brief presentation of this system which is commercially available can be found in the article entitled “New Microkeratomes Still Coming to the Market” by Leah D. Farr in the periodical specialist publication entitled CATARACT & REFRACTIVE SURGERY TODAY EUROPE, issue dated November/December 2007, pages 66, 67.

A variant of the conventional LASIK is Epi-LASIK. Whereas in the case of the LASIK technique the flap is generated by an incision into the stroma, Epi-LASIK avoids a stromal-bed incision for the flap. Instead of this, an epithelial flap is generated along and above Bowman's membrane by guidance of the incision.

Although a generation of the flap by using laser technology has been proposed repeatedly in recent years and has also been implemented in part, the conventional technique of mechanical generation of the flap with a microkeratome continues to have a wide field of application, not least on account of its considerably lower costs in comparison with the laser-assisted variant of flap generation often designated as femto LASIK.

### BRIEF SUMMARY OF THE INVENTION

The invention relates to microkeratome systems that generate a corneal flap with a (at least one) cutting blade. There is no restriction of the applicability of the microkeratome systems being considered here to one of the aforementioned mechanical techniques of flap generation (LASIK, Epi-LASIK). In particular, the microkeratome systems being considered within the scope of the invention are suitable for both techniques.

Differing patients with differing medical indications require the generation of flaps of differing size, differing thickness and differing hinge position. For the purpose of realising differing flap diameters and flap thicknesses, for example, as a rule correspondingly differently dimensioned components are necessary. For example, the flap diameter may have been determined by the inside diameter of a suction ring to be mounted onto the eye. The larger the suction ring (i.e. the larger its inside diameter), the larger the diameter of the flap arising. The flap thickness, on the other hand, may have been determined, for example, by the excess length by which a cutting blade protrudes beyond a levelling face (applanation face) of a cutting head carrying it. Differing cutting heads may accordingly differ by a differing excess length of the cutting blade beyond its levelling face.

Manufacturers of microkeratome systems therefore offer purchasers entire sets of suction rings or/and cutting heads or/and other functional components, in which connection the components in question within each set differ in each instance by the value of at least one characteristic feature. For example, a manufacturer may offer suction rings for the nominal flap diameters 8 mm, 9 mm and 10 mm, as well as cutting heads for the nominal flap thicknesses 100 μm, 130 μm and 150 μm (these numerical values are, of course, to be understood as being purely exemplary and in no way limiting). The operating surgeon then assembles a suitable combination of suction ring, cutting head and, where appropriate, further components, in order to generate a flap having the desired dimensions.

The selection of a suitable combination certainly makes great demands of the experience of the operating surgeon. This is connected, in particular, with the abundance of combinational options and with the fact that many of the combinational options may be more risky than others (for example, very thin flap thicknesses with very large flap diameters). But it also has to be taken into account that in view of the large number of selection options a confusion of the components on the part of the operating surgeon may readily occur.

Against this background, an object of the invention is to make available a microkeratome system for the generation of a flap on the human eye that is able to provide the operating surgeon with better certainty of the suitability of a chosen combination of components of the microkeratome system.

With a view to achieving this object, the invention provides a microkeratome system for the generation of a flap on the human eye, comprising:

a plurality of functionally different, separately interchangeable components with, in each instance, at least one characteristic feature,

a control unit for controlling the operation of the microkeratome system, the control unit having access to stored information that is representative of at least one predetermined combination of the characteristic features of at least a fraction of the components, and

an input device for entering information that is representative of a desired combination of the characteristic features of at least a fraction of the components, wherein the control unit is adapted to compare the desired combination of features with the at least one predetermined combination of features and to bring about a predetermined response, depending on the result of the comparison.

The invention enables the operating surgeon to have a combination of components that is desired by him/her (corresponding to a desired combination of characteristic features of these components) checked for suitability, in particular for permissibility, by the control unit prior to the start of an operation. To this end, via the input device he/she can enter suitable information that specifies the desired combination of features. The desired combination may relate to all the interchangeable components of the microkeratome system that are available and employable in differing variants (i.e. with differing values of their characteristic features). But it is also conceivable that only a combination of a fraction of the interchangeable components which are present overall is the basis for the test for suitability. This is because, to the extent that a certain combination of features of a fraction of the components is already not permissible in principle, for the user it is superfluous to make desired specifications also for the remaining components. For example, it may be that a cutting blade that is suitable for Epi-LASIK can only be used with a specially matched cutting head, equally suitable for Epi-LASIK. On the other hand, if the user enters by way of desired combination a cutting blade that is suitable for Epi-LASIK in conjunction with a cutting head that is merely suitable for conventional LASIK, this already constitutes an impermissible combination, regardless of the concrete choice of features of any further components.

The characteristic features may assume differing forms. It may be a question of numerical values, for example a diameter measurement or thickness measurement, but it may also be a question of properties other than a concrete dimension, for instance a certain structural configuration which, for example, may be represented by a certain code. When a value of a characteristic feature is mentioned here, any realisation of the feature in question is understood thereby, irrespective of whether the feature relates to a dimension, a structural configuration or another unambiguously verifiable peculiarity of the assigned component. Such a peculiarity may, in the case of a motorised drive unit of the microkeratome system, relate, for example, to the number of motors contained in the drive unit (e.g. one motor versus two motors). As mentioned, cutting heads may also differ structurally, for example depending on whether they are to be employed for a LASIK treatment or an Epi-LASIK treatment.

In one embodiment, the components include a suction ring to be mounted onto the eye and capable of being fixed there by reduced pressure, in which case at least one size measure, in particular at least one diameter measure, is assigned to the suction ring as a characteristic feature. Suction rings are conventionally described at least by their inside diameter, frequently also by their outside diameter. The inside diameter and optionally also the outside diameter of the suction ring may then each be utilised by way of characteristic feature.

The components may, in addition, include a cutting blade, in which case the realisation of the cutting blade preferentially serves as a characteristic feature for one of at least two different forms of operation. The aforementioned forms of operation LASIK and Epi-LASIK are, first and foremost, to be mentioned here as possible forms of operation. But, instead of a specification concerning the intended form of operation, the cutting blade may also be defined via certain measurements, for example via a sharpness of the blade (expressed, for example, by a radius of curvature of the cutting edge of the blade). The idea underlying this is that, for Epi-LASIK, use is typically made of a comparatively blunt cutting blade (blunt in comparison with cutting blades for conventional LASIK operations), in order to avoid an unintentional transecting of Bowman's membrane and incising into the stroma.

In another embodiment of the invention, the components may include a cutting head designed for holding a cutting blade and to be brought into engagement with a suction ring, in which case a flap thickness is assigned to the cutting head as a characteristic feature. As mentioned, the excess length of the cutting blade beyond an applanation face of the cutting head may be definitive for the achievable flap thickness. For differing flap thicknesses, differing cutting heads may accordingly be available which differ from one another by the aforementioned excess length.

It has already been mentioned that the cutting head may be differently configured, depending on the form of the operation (e.g. LASIK and Epi-LASIK). Therefore the realisation of the cutting head for one of at least two different forms of operation, in particular LASIK and Epi-LASIK, is preferentially assigned to the cutting head as a further characteristic feature.

The components may furthermore include a motorised drive unit, to which a motor parameter, in particular a number of motors, is assigned by way of characteristic feature.

In a preferred embodiment, the control unit may be adapted to enable or disable the operation of the microkeratome system in the event of conformity of the desired combination of features with a stored predetermined combination of features. To the extent that the stored information relates to permissible combinations of features, the control unit checks an entered desired combination of features as to whether it is permissible. Upon finding a stored identical combination of features, the control unit then enables the microkeratome system for operation. Conversely, it is conceivable that the stored combinations of features constitute impermissible or at least risky combinations. In this case, a disabling of the microkeratome system may be desirable if amongst the stored combinations of features there is one that corresponds to an entered desired combination of features.

Alternatively or additionally to a disabling of the microkeratome system, the control unit may be adapted to bring about the output of at least one of an acoustic and visual indication in the event of conformity of the desired combination of features with a stored predetermined combination of features. Such an indication may warn the operating surgeon, for example, that the combination of components proposed by him/her harbours dangers for the patient.

### DETAILED DESCRIPTION OF PREFERRED EMBODIMENTS

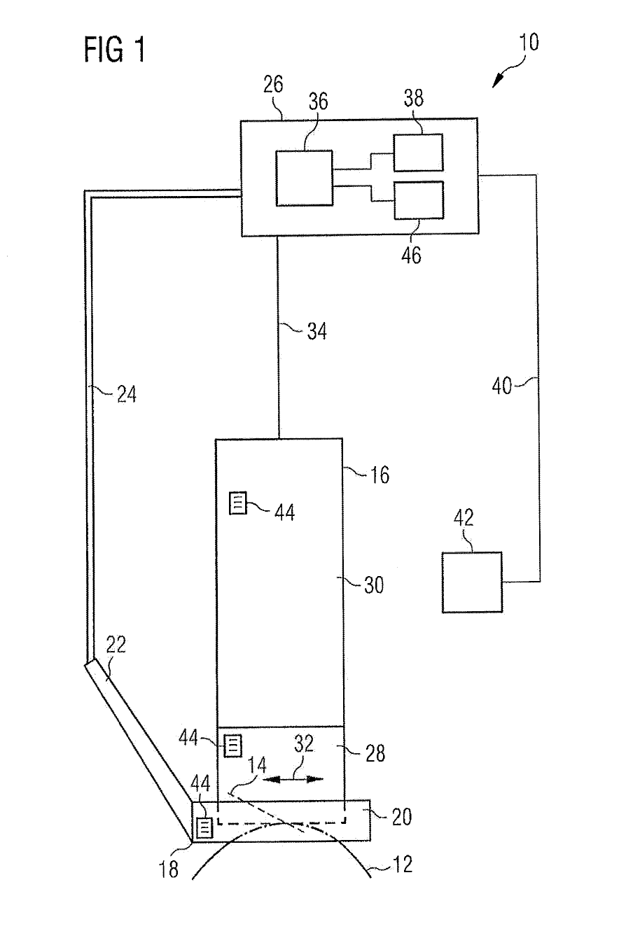

The invention will be elucidated further in the following on the basis of the appended FIG. 1. The latter represents—in greatly simplified, schematic form—an exemplary embodiment of a microkeratome system 10 for generating a corneal flap on a human eye 12. The microkeratome system 10 includes a handpiece 16 equipped interchangeably with a cutting blade 14 and also a suction-ring unit 18 which is composed substantially of a suction ring 20 to be mounted onto the eye 12 and of a connecting piece 22 which is preferentially constructed in one piece with the suction ring 20 and which extends obliquely away from the latter. The connecting piece 22 contains, in a manner not represented but known as such, one or more evacuation ducts which are connected to a corresponding number of evacuation chambers (equally not represented in any detail) formed in the suction ring 20. At least one evacuation chamber of the suction ring 20 serves for aspirating the same onto the surface of the eye 12.

Capable of being attached to the connecting piece 22 is a hose-line arrangement 24 consisting of one or more hose lines, which in turn is capable of being attached to a central control console 26 of the microkeratome system 10 which is constructed as a table-mounted instrument. The control console 26 contains a vacuum-pump arrangement, not represented in any detail, by means of which a vacuum is capable of being generated in each evacuation chamber of the suction ring 20 via the hose-line arrangement 24 and the connecting piece 22.

The handpiece 16 exhibits a cutting head 28 serving as holder for the cutting blade 14, and also a motorised drive unit 30 separately coupled with the cutting head 28. The cutting head 28 is supported on the suction ring 20, guided in a linear direction of motion, by guide structures which are not represented in any detail. For example, the guide structures may include two toothed tracks formed on the suction ring and spaced from one another, into which two pinions arranged on the cutting head 28 and in driving connection with the drive unit 30 engage in intermeshing manner. In this way, a linear reciprocating motion of the cutting head 28 relative to the suction ring 20, and consequently relative to the eye 12, is possible. This reciprocating motion of the cutting head 28 is illustrated in FIG. 1 by a double-headed arrow 32.

The cutting blade 14 is in turn received in the cutting head 28 in laterally mobile manner, ‘laterally’ meaning in a direction perpendicular to the direction of motion 32 of the cutting head 28. For the purpose of lateral oscillation, the cutting blade 14 is also in a driving connection, not represented in any detail, with the drive unit 30.

For the purpose of driving the cutting head 28 and the cutting blade 14, the drive unit 30 may contain a single drive motor (electric motor) or separate motors. A solution with separate motors is shown, for example, in EP 1 757 253 B1; with regard to a one-motor solution, reference may be made, for example, to EP 0 442 156 A1.

An electrical control line 34 connects the control console to the handpiece 16.

Contained in the control console 26 is a microprocessor-assisted control unit 36 which operates in accordance with a control program contained in a memory 38. The control unit 36 controls the operation of the handpiece 16 (more accurately, the operation of the drive unit 30) as well as, preferentially, also the operation of the aforementioned vacuum-pump arrangement. In addition, in the exemplary embodiment that is shown the control console 26 is connected via a further electrical connecting line 40 to a reader 42 which permits the reading of an identifier 44, fitted by the manufacturer (represented, for example, by a bar code), of at least some of the components of the microkeratome system 10. In FIG. 1 such an identifier 44 is present on the drive unit 30, on the cutting head 28 and also on the suction-ring unit 18 (concretely, on the suction ring 20). It will be understood that the cutting blade 14 may also be provided with an identifier of such a type. The identifier 44 contains information that guarantees an unambiguous identification of the component in question, and not only by component type (e.g. cutting head, drive unit, cutting blade, suction ring) but also by concrete realisation of the respective type. The realisation is represented by one or more characteristic features of each component, by which the various realisations of the same component type differ unambiguously from one another.

In the concrete exemplary case the user can choose for the drive unit 30 from amongst at least two variants (realisations): a variant with a single motor, and a variant with two motors. For the suction-ring unit 18 he/she can chose between a plurality of variants which differ in each instance by the inside diameter of the suction ring 20 and consequently by the flap diameter. As far as the cutting head 28 is concerned, the user can choose between two groups of variants: a first group for LASIK operations, and a second group for Epi-LASIK operations. Each group consists, in turn, of several variants which differ by a differing value of the flap thickness achievable with this cutting head. With regard to the cutting blade 14, the user can finally likewise choose between various realisations, a distinguishing feature being the suitability of the cutting blade 14 for LASIK treatments or Epi-LASIK treatments.

The plurality of realisations of the individual components of the microkeratome system 10 creates a still larger plurality of combinational options. In order to facilitate the selection of a suitable combination for the user, in the memory 38 a list of various combinations of components of the microkeratome system 10 is stored, the combinations differing by differing values of at least one characteristic feature of at least one of the components. The list may contain all conceivable combinations of components; but it may also contain only those combinations which are permissible, or alternatively those which are not permissible or are risky. The components considered in a listed combination include, according to a preferred exemplary embodiment, at least the cutting head 28 with the flap thickness by way of characteristic feature and also the suction-ring unit 18 with the inside diameter of the suction ring 20 by way of characteristic feature. Additionally, the drive unit 30 with the number of its motors by way of characteristic feature may have been taken into account in the listed combinations. The configuration of the cutting head 28 for LASIK treatments or for Epi-LASIK treatments may have been taken into account in the listed combinations by way of further characteristic feature of the cutting head 28. Alternatively or additionally, the cutting blade 14 may have been taken into account by way of further component, in particular with regard to its suitability for LASIK treatments or Epi-LASIK treatments.

The aforementioned list of component combinations may have been written into the memory unit 38 by the manufacturer or may be programmed in by a purchaser of the microkeratome system 10, for example prior to putting the same into operation. But in any case it is preferred if within the scope of the planning of a concrete intervention the operating surgeon has no opportunity to change the stored list. Either the list is permanently firmly stored in the memory 38 for this purpose or is modifiable only under certain preconditions, for example only subject to verification of certain access rights. Regardless of this, the control console 26 can enable the user to call up the stored list, in order to display it on a display screen which is not represented in any detail, so that the operating surgeon can, where appropriate, make sure in advance which component combinations are permissible or impermissible.

In the list the components may, for example, be identified by elements of their identifier 44, in particular by those elements which specify the type and the concrete realisation of the respective component.

Within the scope of the planning of the operation, the operating surgeon can, in addition, enter a desired component combination into the central control unit 26. This may, for example, be done by reading in the identifiers 44 of the desired components with the reader 42. Alternatively or additionally, the control console 26 may exhibit a manually operable input unit 46, for instance in the form of a touch-screen, via which the operating surgeon can enter his/her desired combination. Via a suitable menu navigation the operating surgeon can, for example, call up the available realisations of the individual components and select a desirable one from amongst them. As a result, the control console 26 indicates on the touch-screen 46, or on a display screen separate from this, the permissibility or impermissibility of the chosen combination. In the case of an impermissible combination, the control unit 36 can disable the operation of the microkeratome system 10 and, in particular, the operation of the drive unit 30, unless a permissible component combination is selected by the operating surgeon. Cases are also conceivable in which although a desired combination is not impermissible in principle it entails an increased operative risk. In such cases, although the implementation of the operation can be enabled for the operating surgeon, a warning text can be given to him/her on the touch-screen 46 or on another display screen, which draws his/her attention to the dangers by virtue of the chosen combination.

1. A microkeratome system for the generation of a flap on the human eye, comprising:
a plurality of functionally different, separately interchangeable components with, in each instance, at least one characteristic feature,
a control unit for controlling the operation of the microkeratome system, the control unit having access to stored information that is representative of at least one predetermined combination of the characteristic features of at least a fraction of the components, and
an input device for entering information that is representative of a desired combination of the characteristic features of at least a fraction of the components, the control unit adapted to compare the desired combination of features with the at least one predetermined combination of features and to bring about a predetermined response, depending on the result of the comparison.

2. A microkeratome system according to claim 1, wherein the components include a suction ring to be mounted onto the eye and capable of being fixed there by reduced pressure, whereby at least one size measure is assigned to the suction ring as a characteristic feature.

3. A microkeratome system according to claim 2, wherein the at least one size measure includes at least one diameter measure.

4. A microkeratome system according to claim 1, wherein the components include a cutting blade, whereby the realisation of the cutting blade for one of at least two different forms of operation serves as a characteristic feature.

5. A microkeratome system according to claim 4, wherein the forms of operation comprise LASIK and Epi-LASIK.

6. A microkeratome system according to claim 1, wherein the components include a cutting head designed for holding a cutting blade and to be brought into engagement with a suction ring, whereby a flap thickness is assigned to the cutting head as a characteristic feature.

7. A microkeratome system according to claim 6, wherein the realisation of the cutting head for one of at least two different forms of operation, preferably LASIK and Epi-LASIK, is assigned to the cutting head as a further characteristic feature.

8. A microkeratome system according to claim 1, wherein the components include a motorised drive unit, to which a motor parameter is assigned by way of characteristic feature.

9. A microkeratome system according to claim 8, wherein the motor parameter is a number of motors included in the motorised drive unit.

10. A microkeratome system according to claim 1, wherein the control unit is adapted to perform one of enabling and disabling the operation of the microkeratome system in the event of conformity of the desired combination of features with a stored predetermined combination of features.

11. A microkeratome system according to claim 1, wherein the control unit is adapted to bring about the output of at least one of an acoustic and a visual warning message in the event of conformity of the desired combination of features with a stored predetermined combination of features.
